(19) United States
(12) Patent Application Publication
(10) Pub. No.: US 2002/0028441 A1
(43) Pub. Date: Mar. 7, 2002

(54) DETECTION OF MOLECULES AND MOLECULE COMPLEXES

(76) Inventors: RAINER HINTSCHE, BERLIN (DE); MANFRED PAESCHKE, BASDORF (DE)

Correspondence Address: PATRICIA D. GRANADOS, HELLER EHRMAN WHITE & McAULIFFE LLP, 815 CONNECTICUT AVENUE, N.W., SUITE 200, WASHINGTON, DC, 20006, US

(21) Appl. No.: 09/142,660

(22) PCT Filed: Mar. 12, 1997
(86) PCT No.: PCT/DE97/00494

(30) Foreign Application Priority Data
Mar. 14, 1996 (DE) 196 10 115.8

(51) Int. Cl. C12Q 1/68; G01N 33/53; C12P 19/34; G01N 33/00; G01L 1/20; C07K 1/26; C02F 1/469; G01F 1/64; G01N 33/566; B01D 57/02

(52) U.S. Cl. 435/6; 435/91.1; 435/183; 435/7.1; 204/456; 436/94; 436/501

(57) ABSTRACT

The invention concerns a process for detecting molecules or molecule complexes. A measurement probe is brought into contact with an ultramicroelectrode arrangement comprising at least two electrode structures configured in such a way that the distances between the different structures lie in the ultramicro range; an alternating electrical field is created by application of an electrical potential; and the current or potential fluctuations caused by species present or created in the measurement probe are measured.

The invention relates to a method for the detection of molecular species, and to an electronic sensor therefor. Electronic sensors of this type, also referred to as ultra-microelectrode arrays, can be used for chemical analysis and process control in a variety of fields, such as the health service, biotechnology, environmental protection and the chemical industry. They represent a comparatively simple measuring system which measurably registers the binding or attachment of molecules in the area close to the electrodes.

Hitherto known are optical sensors which make it possible to detect binding effects or the attachment of molecules in thin layers, amongst other things, using the evanescent wave [cf. Feldman et al., Biosens. & Bioelectron., 10 (1995) 423] or light-reflection [cf. Domenici et al., Biosen. & Bioelectron., 10 (1995) 371 or Brecht, Gauglitz, Biosen. & Bioelectron., 10 (1995), 923] or surface plasmon resonance [cf. Häuseling et al., Langmuir, 7 (1991) 1837 or U. Jönsson et al., BioTechniques 11 (1991), 620] principles.

For the direct electrical reading of binding events of this type, a potentiometric measurement method [cf. Bergfeld, Biosen. & Bioelectron., 6 (1991), 55], a capactive measurement method [cf. Swietlow, Electroanalysis, 4 (1992), 921] and an impedimetric measurement method [cf. Knichel et al. Sens. & Act. B 28, (1995), 85] have already been described. Electrode arrangements based on the EIS principle (EIS: Electrolyte-insulator-semiconductor) have also been proposed [cf. Schyberg et al. Sens. & Act. B 26-27 (1995) 457 or Souteyrand et al. Sens. & Act. B 20, (1994) 63], the insulator acting as a coupling and relay element.

In these electrochemical or measurement arrangements, electrodes that are spatially far removed from one another are used to register molecules in the thin boundary layer close to the electrodes, but these are negatively affected in a variety of ways by a comparatively large amount of electrolytes and other substances between the electrodes.

Applications are also known in which thin molecular layers have been deposited as a gate between the drain and source of transistors and provide information regarding the organic layer [cf. Kruse et al. Sens. & Act. B 6 (1992), 101 or Uhe et al. Electroanalysis, 6 (7) (1994), 543].

A fact common to all these described electrical methods with electrodes is that they do not have any arrangements approaching molecular dimensions; in all of these applications, the lengths typical of the sensors, for example between the measuring, reference and working electrodes, are orders of magnitude away from molecular dimensions.

The object of the invention is to provide a method using an electronic sensor which permits the detection of molecules and molecule complexes at higher detection sensitivity with comparatively lower system outlay.

The way in which this object is achieved according to the invention is described in claim 1. The further claims present preferred refinements.

According to the invention, the method for the detection of molecules and molecule complexes is carried out with an arrangement which has an ultra-microelectrode array whose electrode structures are arranged so closely next to one another that they approach the size of large molecule complexes, for example immunoproteins or DNA molecules. Use is, in particular, made of the effect that it is possible for alternating electric fields to be produced between closely neighboring electrodes and the resulting current is predominantly affected by the detected molecules and molecule complexes in the area close to the electrodes. There is in this case a relatively free choice over the shape and fine structure of the electrodes, while the minimum spacing of the electrodes themselves should typically be less than 3 μm, preferably 1 μm.

The way in which the current is affected may involve diffusion, attachment or binding of the species to be measured. Through this way of generating the field and of taking measurements, in particular using impedance spectroscopy, the invention achieves the result that electrolyte molecules and other substances in a sample to be measured have only a slight effect on the electric field existing between the electrodes, and do not therefore interfere with the measurement.

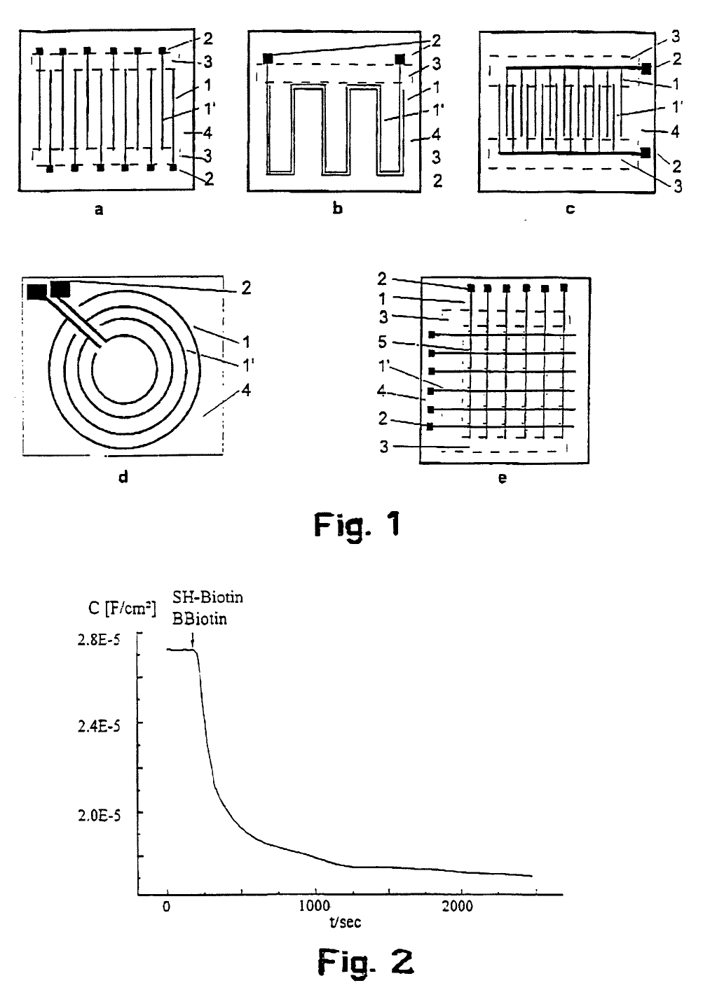

A multiple arrangement of this kind of fine-structured ultra-microelectrode array advantageously leads to amplification of the effect described above, in which measurements of the same type are taken sequentially or in parallel using a suitable measuring technique (for example impedance measurement bridges). The ultra-microelectrode arrays may consist of thin layers of noble metals such as gold, platinum or iridium, or alternatively carbon materials, or may contain these materials (claim 16). They are particularly advantageously applied to planar insulating support materials such as silicon compounds, glass, ceramic or organic polymers, but may also, for planarization and mechanical support, be buried or incorporated in these materials (claim 17). Two mutually insulated ultra-microelectrodes can be brought together optimally, as represented in FIG. 1, for example using bands or parallel strips or meandering and round or coiled structures, as well as using finger-like interdigital arrangements at distances of preferably less than 1 μm. In relation to this, FIG. 1 gives arrangement examples a to d (see below). The electrodes are preferably uncovered in the direction of the measurement area.

One particular refinement of the arrangement of the ultra-microelectrode array which may be provided is to stack an electrode array with one or more others and to insulate the crossover points from one another using insulation layers (claim 19). It is in this way possible for the electrodes to be arranged at distances of only a few nm from one another, with the insulation layer defining the minimum spacing (FIG. 1e). One fact common to all the ultra-microelectrode array arrangements is that they must be properly insulated from one another so that two, three or more ultra-microelectrode arrays can have direct and/or alternating current applied to them individually or in groups, electrically independently through an insulated supply lead on the chip (claim 20). The materials used for the insulation (for example plastics or inorganic compounds such as silicon oxides, nitrides and ceramic materials) need to be inert over the working period with respect to the diluents or solvents (often water) used in the sample. The term “solvent” is intended to mean reaction liquids in which it is possible for the molecules to bind, become attached or diffused. The sample to be measured need not, however, necessarily be liquid, and other states are also possible. The processes to be measured may thus also take place in a gel.

Between the ultra-microelectrodes, the electric field employed for detection may be produced by alternating current with frequencies of between 1 mHz and 10 MHz and amplitudes of about 10 mV and 50 mV. In this case, potentials of between 0 V and +/−5 V are chosen.

The present method even makes it possible to register complex reaction processes, and therefore affords enhanced possibilities for use. The penetration of molecules into the region close to the electrodes with the field which is built up (for example by diffusion) or the arrangement of molecules in this region, which may for example take place through so-called “self assembling” or else through complexing, alter both the real and imaginary parts of the complex impedance, and may be measured independently of time—for example after the events have ceased to take place—as well as with the phase angle, if necessary, but it may also be measured as a function of time, that is to say on the basis of the progress of the binding event or the diffusion (claims 3 and 4). For a complete impedance spectrum, the entire frequency range is measured and evaluated. The use according to the invention of only individual selected frequencies or frequency ranges, which are maximally affected, is particularly advantageous. This makes it possible to design miniaturized detection systems.

When use is made of the ultra-microelectrode arrays in liquids or the like, it is also possible, in addition to their measuring process—or alternatively in pauses between measurements—for direct-current components to be superimposed or applied (claim 6). These may, for example, induce electrochemical reactions such as oxidations or reductions of electrically active molecules, with processes of this type being measured simultaneously or sequentially with the impedance measurements (claim 7). According to the invention, this permits a combination of electrical and electrochemical measurements with the same sensor arrangement (ultra-microelectrode array).

According to the invention, the method may be carried out for the detection of molecules and molecule complexes by making the molecules which are to be measured bind to the actual microelectrode surfaces. This binding may be physical (adsorption) or chemical. For the latter case, the self-assembling methods are particularly well-suited, which make it possible, for example, to bind monomolecular thiol compounds on gold electrodes and measure them. This method is universally applicable for a large number of molecules, and not only for those which have, or may be provided with, a thiol group.

A second selective method for making molecules and molecule complexes adhere to the conductive microelectrode surfaces is the known method of electropolymerization (claim 9). In this case, each electrode may be modified individually, in groups or in parallel, on its surface with electropolymers, for example made up of the monomer molecules streptavidin, pyrrole, aniline, vinyl ferrocene or other electrically polymerizable substances. The binding of compounds of this type in monomolecular or multimolecular layers on the electrodes changes the impedance spectrum or individual frequencies in a very characteristic fashion, and can therefore be measured as a function of time or after completion of the reaction.

Further, the impedance spectrum may also be measurably changed if the molecules are positioned in the gaps between electrodes instead of on the electrodes (claim 10). This positioning may be carried out, for example, by chemical binding (for example to silicon dioxide) or by adhesion or by reactions such as condensation reactions, for example silanizing. In order to coat the entire surface of the electrode array, that is to say the electrodes themselves as well as the gaps between electrodes, the known Langmuir-Blodget method may be employed (Tachibana Matsumoto, Advanced Materials Ab. 11 (1993), 5/796-803) with which, for example, lipids or phthalocyanines can be arranged in layers by pulling monomolecular films.

According to a further variant of the method according to the invention for the detection of molecules and complexes, the concentration of molecules in the layer close to the electrodes may be altered by diffusion, and the alteration may be measured. This can be done both using chemically/physically related changes in concentration, and by applying an electric potential which produces a diffusion gradient. It is further possible to bring about and measure the production of specific molecules, for example by enzymes, in the area close to the electrodes.

According to the invention, in a preferred refinement, the method for the detection of molecules and molecule complexes comprises the measure that the molecular layers produced beforehand on the electrode arrays are or will be provided with chemical bonding groups that can bind further molecules by a chemical reaction or complexing (claim 11). It is in this way possible to monitor binding events of this type with high sensitivity. If, for example, a complexing agent of low molecular weight such as biotin is bound to the electrode via a thiol functional group, then this biotin may subsequently be complexed with a complexing partner of fairly high molecular weight, for example streptavidin, to which an arbitrary number of further molecules can be bound.

One particularly important and very widely usable application of the present invention is immunodetection (claim 12). In this case, molecular layers are built up on the ultra-microelectrode array using the sandwich principle of an antibody/antigen immune reaction. In order to detect antibodies in the sample to be measured, haptens (antigens with low molecular weight) or other antigens (often proteins), for example, may for this purpose be bound to the microelectrode arrays. In this way, the specific complexing between the firmly anchored antigens and the antibodies found in the sample to be measured lead to specific antibody detection. In reversal of this principle, it is also possible for the antibodies to be bound to the electrodes and for haptens or the like to be detected from the sample to be measured. The antigen may also be a virus protein with fairly high molecular weight, which is firmly bound to the microelectrode array and makes it possible to measure antibodies from the sample to be measured. Variants of this method include the use of polyvalent antibodies with which it is possible to construct and measure threefold or higher molecule complexes.

A further refinement of the method according to the invention is provided if the ultra-microelectrode array is used for the electrical reading of hybridization processes in nucleic acid chemistry (claim 13). Applications in genetic engineering can be produced by binding nucleotides via thiol bonds or the like to the electrode structures and registering the binding of complementary nucleic acid components using the method according to the invention. This detection can be varied by making additional attachments of nucleic acids, for example to form triple DNA, or the additional incorporation of complexing molecules in double or triple helices accessible to measurement as binding events (claim 14). For this complexing or incorporation, use may advantageously also be made of metal complexes which bring about a particularly intensive electrical change in the field close to the electrodes.

The measurement principle, and the change in the electric field, make it possible in principle to distinguish the structure and nature of molecules by means of quantitative analysis of the impedance spectrum. Differentiation according to type and size of the molecules is possible through quantitative evaluation and, in particular, by calibration of the impedance spectra using known molecular species.

The invention will be explained below with reference to several figures and an example.

FIG. 1 shows possible arrangements of the ultra-microelectrode arrays;

FIG. 2 shows the adsorption of SH-biotin;

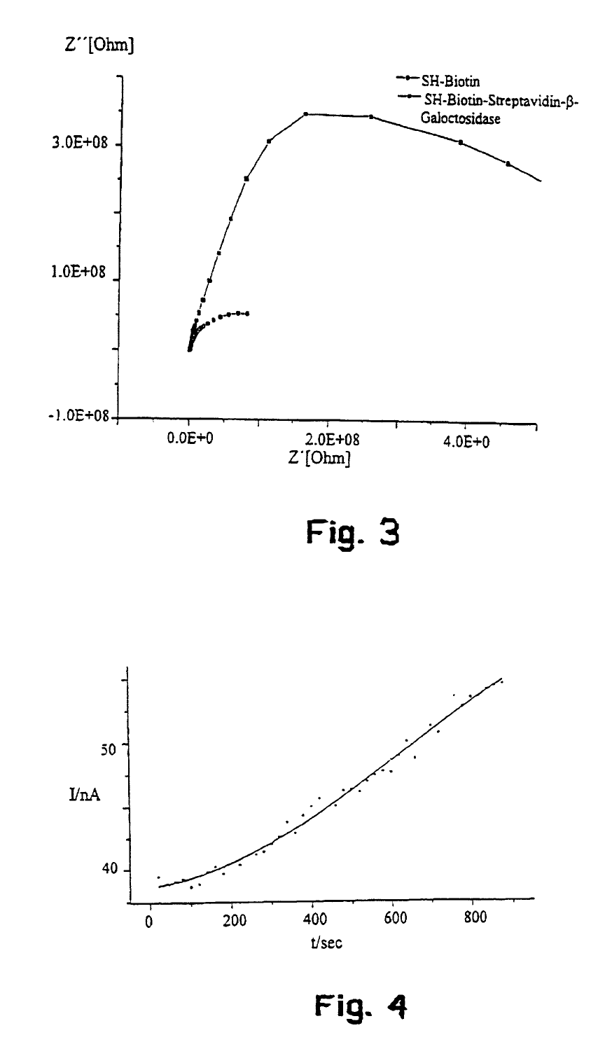

FIG. 3 shows Nyquist plots of an electrode modified with SH-biotin and one additionally complexed with streptavidin;

FIG. 4 shows the amperometric section of p-aminophenol.

FIG. 1 shows various possible arrangements of ultra-microelectrode arrays. In this case

1a is a parallel arrangement in the form of strips;

1b is a parallel arrangement in the form of meanders;

1c is a finger-like interdigital arrangement;

1d is a circular parallel arrangement;

1e is a circularly stacked and mutually insulated arrangement;

Very much like the arrangement in FIG. 1d is the arrangement of the electrodes as coils running parallel.

The mutually insulated ultra-microelectrodes 1 and 1′, with their contacts to the electrical connection 2 and to the insulation layers (for example silicon nitride) 3 on the chip are arranged on a planar support (for example a silicon chip) 4. In the multilayer arrangement in FIG. 1e, the electrode plane 1 is insulated from the electrode plane 1′ by intermediate insulation 5.

### Illustrative Embodiment

An interdigital gold electrode array, structured according to FIG. 1c, has an electrode width of 1 μm and an electrode spacing of 0.7 μm. The electrodes are modified with a 1 ml, 10 mmol/l SH-biotin solution by means of self-assembling.

FIG. 2 represents the adsorption of 10 mmol/l SH-biotin in a 0.1 mol/l sodium buffer solution as a capacitance/time plot for an applied potential of 50 mV and an additionally imposed amplitude of 10 mV at a pair of interdigital gold electrodes. The electrode capacitance decreases after the addition of SH-biotin to the solution. After about 2000 seconds, the surface of the gold is fully covered with -S-biotin. After 10 min of washing the electrode in 0.1 mol/l sodium buffer solution, the adsorbed monomolecular molecular layer is complexed in a subsequent step with streptavidin by dipping the modified electrode for 2 hours in a 50 U/ml solution. After the β-galactosidase-streptavidin modification, the electrode was rinsed for 10 min in 0.1 mol/l sodium buffer solution and subsequently secured in a measuring cell.

FIG. 3 shows so-called Nyquist plots for a potential of 50 mV, an amplitude of 10 mV and a frequency range of between 2×10⁻³ Hz and 1×10⁶ Hz, measured as two-pole impedance. Curve I represents the electrode modified with SH-biotin, and curve II the same electrode after additional complexing of the SH-biotin with β-galactosidase-streptavidin. The change in the impedance shows the disturbance of the dielectric between the electrodes by the complexed molecule, and further represents completed binding between the biotin and the streptavidin-enzyme complex.

The enzyme β-galactosidase on streptavidin is used independently as combined amperometric detection of the binding of the β-galactosidase-streptavidin to the SH-biotin. This detection is carried out with the function of the β-galactosidase, the enzymatic conversion of 5 mmol/l p-aminophenyl-β-D-galactopyranoside (p-APG) to p-aminophenol, by means of an amperometric oxidation-reduction of the p-aminophenol.

FIG. 4 shows the amperometric detection of p-aminophenol on the same electrodes with an oxidation potential of 250 mV and a reduction potential of −50 mV relative to an Ag/AgCl reference electrode, after the addition of 5 mmol/l p-APG in 0.1 mol/l sodium buffer solution to the measuring cell. The continuous conversion of p-APG to p-aminophenol, which is represented by the linear rise in the current, indicates that the enzyme increases the p-aminophenol concentration in the measuring chamber.

1. Method for the detection of molecules or molecule complexes,
a sample to be measured being brought into contact with an ultra-microelectrode arrangement which has at least two electrode structures that are arranged relative to one another such that the distances between the various structures lie in the ultra-micro range,
an alternating electric field being produced by application of an electric potential, and
the changes in current or potential, which are caused by species present or created in the sample to be measured, being measured.

2. Method according to claim 1, in which the field changes are measured using impedance spectroscopy.

3. Method according to claim 1 or 2, in which the detuning of the electric field, which is caused by species present or created in the sample to be measured, is measured independently of time or as a function of time by measuring the capacitive and/or resistive components and/or the phase angle.

4. Method according to one of claims 1 to 3, in which the molecules or molecule complexes are detected by virtue of their binding or attachment or diffusion.

5. Method according to one of claims 1 to 4, a plurality of electrode arrangements being stacked, and the crossover points being insulated from one another by insulation layers, and the measurements being taken sequentially, in parallel or simultaneously.

6. Method according to one of claims 1 to 5, characterized in that the alternating electric field is superimposed or excited with a direct-current component.

7. Method according to claim 6, amperometric oxidations or reductions or redox recycling of molecules having electrically active groups or of redox mediators being measured in the sample to be measured.

8. Method according to one of claims 1 to 7, in which species to be measured self-assemble on the active electrode surfaces and are measured in the bound state.

9. Method according to one of claims 1 to 8, in which molecules are bound on the electrode surfaces by electropolymerization and are measured in the bound state.

10. Method according to one of claims 1 to 9, molecules being fixed in the gaps between electrodes and/or on the entire surface of the electrodes by physical or chemical binding, and being measured.

11. Method according to one of claims 8 to 10, a first fixed molecular layer containing a bonding group which, itself or through a difunctional reagent, binds a second molecular layer, which may in turn bind others, and these events or their reverse being measured.

12. Method according to claim 11, in which the first molecular layer contains complexing groups which bind their complementary binding partner these events or their reverse being measured.

13. Method according to claim 11 or 12, in which the first molecular layer is a deoxiribonucleic-acid or ribonucleic-acid component which binds a complementary molecule strand by hybridization, this event or its reverse being measured.

14. Method according to claim 13, in which the molecular arrangement binds a further nucleic acid component or a complexing or intercalating molecule, and this event or its reverse is measured.

15. Method according to one of claims 1 to 14, in which the molecules or molecule complexes are detected in that they differ by size and/or type.

16. Method according to one of claims 1 to 15, in which the active electrode surfaces consist of gold, platinum, iridium or other noble metals, of carbon materials or of other conductive materials or of combinations thereof.

17. Method according to one of claims 1 to 16, in which the electrodes are applied to, or are incorporated in, silicon compounds, glass, ceramic, organic polymers or other insulating materials.

18. Method according to one of claims 1 to 17, in which, by coating on a substrate or embedding in such, the electrodes are arranged as bands or strips or circular structures or interdigital arrangements in the micrometer or submicrometer separation from one another.

19. Method according to one of claims 1 to 18, in which at least some of the electrodes are arranged as multilayer structures that are insulated from one another and, if appropriate, intersect.

20. Method according to one of claims 1 to 19, in which the active electrode surfaces can have direct and/or alternating current applied to them, individually or in groups, via insulated supply leads and/or electronic components.
